(19) United States
(12) Patent Application Publication
(10) Pub. No.: US 2025/0143807 A1
(43) Pub. Date: May 8, 2025

(54) PROVISION OF SURGICAL GUIDANCE BASED ON AUDIOVISUAL DATA AND INSTRUMENT DATA

(71) Applicants: COVIDIEN LP, Mansfield, MA (US); DIGITAL SURGERY LIMITED, London (GB)

(72) Inventors: Robert M. Nostrant, Berthoud, CO (US); Susan L. Roweton, Lafayette, CO (US); Petros Giataganas, London (GB); Danail V. Stoyanov, London (GB); Carole RJ Addis, London (GB); Tom K. Matthews, Tunbridge Wells (GB); Anthony L. Ceniccola, Hamden, CT (US); Christopher K. Evans, Southington, CT (US); Kasey A. Grim, Lafayette, CO (US); Drew Robert Seils, Guilford, CT (US); Christopher Switalski, Glastonbury, CT (US); Benjamin J. Noe, Erie, CO (US); Patrick Dale Mozdzierz, Glastonbury, CT (US); Michael S. Gallie, Branford, CT (US); Scott J. Prior, Branford, CT (US); Heather I. Tuccolo, Salem, NH (US); Sheldon K. Hall, London (GB); James G. Pappas, Longmont, CO (US); Scott M. Kilcoyne, Niwot, CO (US)

(21) Appl. No.: 18/833,570

(22) PCT Filed: Jan. 28, 2023
(86) PCT No.: PCT/EP2023/052097

§ 371 Date: Jul. 26, 2024

(30) Foreign Application Priority Data
Jan. 28, 2022 (GR) 20220100087

(51) Int. Cl. A61B 34/00; A61B 1/00; A61B 1/04; A61B 17/00; A61B 17/068; A61B 90/00

(52) U.S. Cl. CPC A61B 34/25; A61B 1/00045; A61B 1/04; A61B 17/00234; A61B 17/068; A61B 2017/00411; A61B 2034/254; A61B 2090/0803

(57) ABSTRACT

A system is provided that includes a memory device and one or more processors coupled with the memory device. The one or more processors are configured to determine, autonomously, one or more phases in a surgical procedure based on a video stream of the surgical procedure. The one or more processors are further configured to identify one or more usages of a surgical instrument used during the surgical procedure. The one or more processors are configured to display a chart of the one or more usages. The chart divides the one or more usages according to the one or more phases respectively, and a representation of each of the one or more usages indicates a duration of each usage.

### BACKGROUND

The present disclosure is generally related to computing technology, particularly to improvements to computer-assisted surgical systems that facilitate provision of surgical guidance based on audiovisual data and instrument data.

Computer-assisted surgery (CAS) includes the use of computer technology for surgical planning, guiding or performing surgical interventions, and postoperative analysis. CAS, in some aspects, can include robotic surgery. Robotic surgery can include a surgical instrument that performs one or more actions in relation to an action performed by medical personnel, such as a surgeon, an assistant, a nurse, etc. Alternatively, or in addition, the surgical instrument can be part of a supervisory-controlled system that executes one or more actions in a pre-programmed or pre-trained manner. Alternatively, or in addition, the medical personnel manipulates the surgical instrument in real-time. In yet other examples, the medical personnel carries out one or more actions via a platform that provides controlled manipulations of the surgical instrument based on the personnel's actions. In some aspects, data captured during the CAS, which includes but is not limited to instrument timing, instrument metrics, audio, video, images, operational notes, medical records, etc., are analyzed post-surgery.

### BRIEF DESCRIPTION

According to one or more aspects, a system includes a memory device, and one or more processors coupled with the memory device. The one or more processors determine, autonomously, one or more phases in a surgical procedure based on a video stream of the surgical procedure. The one or more processors identify one or more usages of a surgical instrument used during the surgical procedure. The one or more processors display a chart of the one or more usages, wherein the chart divides the one or more usages according to the one or more phases respectively, and a representation of each of the one or more usages indicates a duration of each usage. In some aspects, usage includes activation of the surgical instrument. Alternatively, or in addition, the usage can include reloading of the surgical instrument (e.g., stapler). Alternatively, or in addition, the usage can include firing of the surgical instrument (e.g., stapling). Alternatively, or in addition, the usage can include incision, dividing, clamping, or other actions performed using the surgical instrument.

In one or more examples, the video stream of the surgical procedure is analyzed by a first device to determine and output the one or more phases in the surgical procedure, and wherein the one or more usages of the surgical instrument are identified by a second device based on electrical energy applied to the surgical instrument.

In one or more examples, the usage identified based on an amount of electrical energy provided to the surgical instrument.

In one or more examples, the video stream of the surgical procedure captured by an endoscopic camera from inside a body of a subject of the surgical procedure.

In one or more examples, a visual attribute of the representation of each of the one or more usages is based on a type of the one or more usages.

In one or more examples, the one or more processors display a number of different types of usages detected based on the electrical energy provided to the surgical instrument.

In one or more examples, the chart is user-interactive, and wherein an interaction with a first representation corresponding to a first usage displays a video segment of the surgical procedure comprising the first usage being performed.

In one or more examples, the one or more processors playback the video stream of the surgical procedure, and wherein a user-interface element displays a timeline depicting one or more timepoints in the video stream at which the one or more usages are performed.

In one or more examples, the one or more timepoints are rendered based on a type of the one or more usages respectively.

In one or more examples, audio data corresponding to the one or more usages is generated during the playback of the video stream.

In one or more examples, the one or more processors display a list of the one or more phases in the surgical procedure, wherein an entry corresponding to a first phase from the one or more phases includes a user-interface element comprising a timeline depicting the one or more usages performed for the first phase.

In one or more examples, the representation of each of the one or more usages indicates a user that performed the usage.

In one or more examples, the one or more processors depict a comparison of usages performed by a first user and a second user.

In one or more examples, the representation of each of the one or more usages indicates an anatomical attribute of the subject of the surgical procedure, the anatomical attribute comprising a body mas index, a tissue thickness, and a gender.

According to one or more aspects, a method includes determining, autonomously, one or more phases in a surgical procedure based on a video stream of the surgical procedure. The method further includes identifying one or more usages of a surgical instrument used during the surgical procedure based on energy supplied to the surgical instrument. The method further includes displaying a chart of the one or more usages and a user-interaction with a representation of each of the one or more usages causes a corresponding portion of the video stream to be played back.

In one or more examples, the chart groups the one or more usages according to the one or more phases respectively.

According to one or more aspects, a computer program product includes a memory device with computer-readable instructions stored thereon, wherein executing the computer-readable instructions by one or more processing units causes the one or more processing units to perform the above method.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 shows a computer-assisted surgical system according to one or more aspects;

FIGS. 2-8 depict example user-interactive reports of a surgical procedure according to one or more aspects;

FIG. 9 depicts an example user-interactive report of a comparison of surgical procedures according to one or more aspects;

FIG. 10 depicts an example user-interactive report of a comparison of surgeons according to one or more aspects;

FIGS. 11-12 depict example user-interactive reports summarizing the usage of a computer-assisted surgical system according to one or more aspects; and

FIGS. 13-16 depict example user-interactive reports of a surgical procedure according to one or more aspects.

The diagrams depicted herein are illustrative. There can be many variations to the diagram, or the operations described therein without departing from the spirit of the invention. For instance, the actions can be performed in a differing order, or actions can be added, deleted, or modified. Also, the term “coupled”, and variations thereof describe having a communications path between two elements and do not imply a direct connection between the elements with no intervening elements/connections between them. All of these variations are considered a part of the specification.

### DETAILED DESCRIPTION

Exemplary aspects of technical solutions described herein relate to, among other things, devices, systems, methods, computer-readable media, techniques, and methodologies for using machine learning and computer vision to improve computer-assisted surgical systems. In one or more aspects, the structures are predicted dynamically and substantially in real-time as the surgical data is being captured and analyzed by technical solutions described herein. A predicted structure can be an anatomical structure, a surgical instrument, etc. Exemplary aspects of technical solutions described herein further facilitate generating augmented views of surgical sites using semantic surgical representations based on the predictions of the one or more structures in the surgical data.

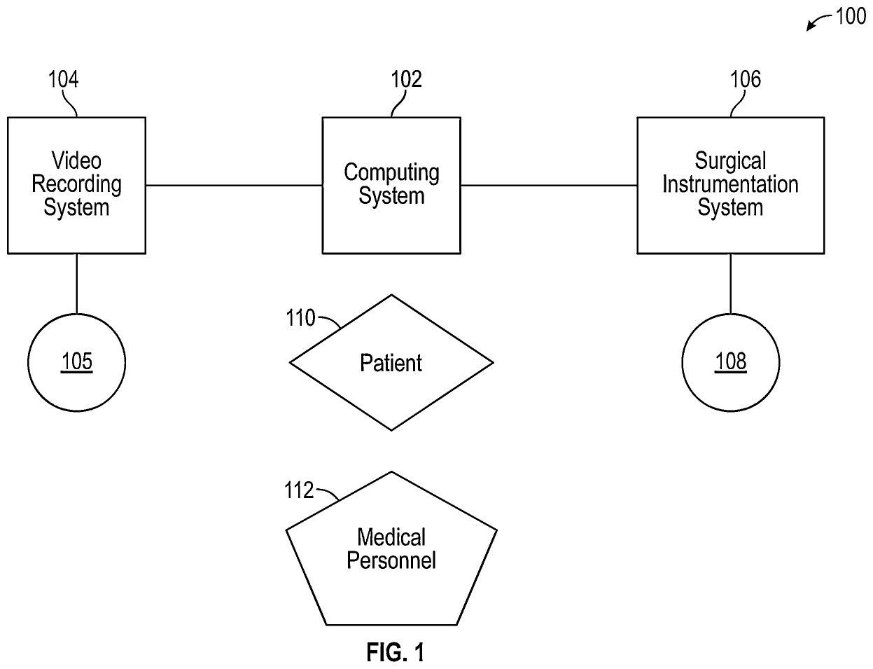

FIG. 1 depicts an example CAS system according to one or more aspects. The CAS system 100 includes at least a computing system 102, a video recording system 104, and a surgical instrumentation system 106.

Actor 112 can be medical personnel that uses the CAS system 100 to perform a surgical procedure on a patient 110 (e.g., a subject of the surgical procedure). Medical personnel can be a surgeon, assistant, nurse, administrator, or any other actor that interacts with the CAS system 100 in a surgical environment. The surgical procedure can be any type of surgery, such as but not limited to cataract surgery, laparoscopic cholecystectomy, endoscopic endonasal transsphenoidal approach (eTSA) to resection of pituitary adenomas, or any other surgical procedure. In other examples, the actor 112 can be a technician, an administrator, an engineer, or any other such personnel that interacts with the CAS system 100. For example, the actor 112 can record data from the CAS system 100, configure/update one or more attributes of the CAS system 100, review past performance of the CAS system 100, repair the CAS system 100, etc.

A surgical procedure can include multiple phases, and each phase can include one or more surgical actions. A “surgical action” can include an incision, a compression, a stapling, a clipping, a suturing, a cauterization, a sealing, or any other such actions performed to complete a phase in the surgical procedure. A “phase” represents a surgical event that is composed of a series of steps (e.g. closure). A “step” refers to the completion of a named surgical objective (e.g., hemostasis). During each step, certain surgical instruments 108 (e.g., forceps) are used to achieve a specific objective by performing one or more surgical actions.

The surgical instrumentation system 106 provides electrical energy to operate one or more surgical instruments 108 to perform the surgical actions. The usage of the surgical instruments 108 can be monitored based on the electrical energy provided. The usage can include an activation, operation, and other actions performed using the surgical instruments 108. Alternatively, or in addition, the usage can include reloading of the surgical instrument 108 (e.g., stapler). Alternatively, or in addition, the usage can include firing of the surgical instrument 108 (e.g., stapling). Alternatively, or in addition, the usage can include incision, dividing, clamping, or other actions performed using the surgical instrument 108.

The electrical energy triggers a usage in the surgical instrument 108. The electrical energy can be provided in the form of an electrical current or an electrical voltage. The usage can cause a surgical action to be performed. The surgical instrumentation system 106 can further include electrical energy sensors, electrical impedance sensors, force sensors, bubble and occlusion sensors, and various other types of sensors. The electrical energy sensors can measure and indicate an amount of electrical energy applied to one or more surgical instruments 108 being used for the surgical procedure. The impedance sensors can indicate an amount of impedance measured by the surgical instruments 108, for example, from the tissue being operated upon. The force sensors can indicate an amount of force being applied by the surgical instruments 108. Measurements from various other sensors, such as position sensors, pressure sensors, flow meters, can also be input. For example, an articulated angle of a stapler can be measured by such sensors. Further yet, a type of staple being used, amount of compression being applied (e.g., stapler, clamp, etc.), can also be measured and recorded. Amount of energy being supplied to the surgical instrument 108 can indicate the amount of pressure being applied in one or more aspects. The amount of energy, in some aspects in combination with measurements from other sensors, can indicate the type of usage of the surgical instrument 108. The surgical instrumentation system 106 can further include electrical energy sensors, electrical impedance sensors, force sensors, bubble and occlusion sensors, and various other types of sensors. It should be noted that the sensors and data are provided as examples herein, and aspects of the technical solutions described herein should not be limited to only the examples provided herein. Several types of data can be received, analyzed, generated, and displayed via one or more dashboards/user-interfaces described herein. For example, the user-interfaces can include data from device operation such as motor speeds, motor position, motor current draw, motor controller settings, temperature, device battery levels, accelerometer readings, user inputs (key activations), device display status (what screen the device is displaying), duty cycles, and internal system communications.

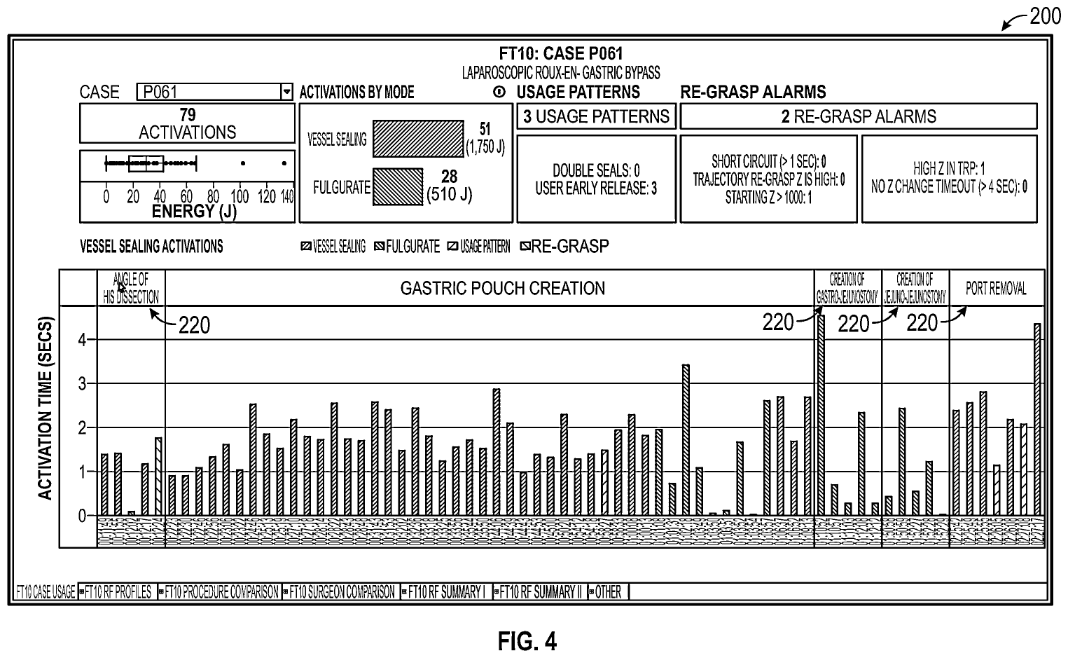

The video recording system 104 includes one or more cameras, such as operating room cameras, endoscopic cameras, etc. The cameras capture video data of the surgical procedure being performed. The video recording system 104 includes one or more video capture devices that can include cameras placed in the surgical room to capture events surrounding (i.e., outside) the patient being operated upon. The video recording system 104 further includes cameras that are passed inside (e.g., endoscopic cameras) the patient to capture endoscopic data. The endoscopic data provides video, images of the surgical procedure (e.g., FIG. 4).

The computing system 102 includes one or more memory devices, one or more processors, a user interface device, among other components. The computing system 102 can execute one or more computer-executable instructions. The execution of the instructions facilitates the computing system 102 to perform one or more methods, including those described herein. The computing system 102 can communicate with other computing systems via a wired and/or a wireless network. In one or more examples, the computing system 102 includes one or more trained machine learning models that can detect and/or predict features of/from the surgical procedure that is being performed, or has been performed earlier. Features can include structures such as anatomical structures and surgical instruments (108) in the surgical procedure. Features can further include events such as phases and actions in the surgical procedure. Features that are detected can further include actor 112, patient 110. Based on the detection, the computing system 102, in one or more examples, can provide recommendations for subsequent actions to be taken by actor 112. Alternatively, or in addition, the computing system 102 can provide one or more reports based on the detections. The detections by the machine learning models can be performed in an autonomous or semi-autonomous manner.

The machine learning models can include artificial neural networks, such as deep neural networks, convolutional neural networks, recurrent neural networks, encoders, decoders, or any other type of machine learning models. The machine learning models can be trained in a supervised, unsupervised, or hybrid manner. The machine learning models can be trained to perform detection and/or prediction using one or more types of data acquired by the CAS system 100. For example, the machine learning models can use the video data captured via the video recording system 104. Alternatively, or in addition, the machine learning models use the surgical instrumentation data from the surgical instrumentation system 106. In yet other examples, the machine learning models use a combination of the video and the surgical instrumentation data.

Additionally, in some examples, the machine learning models can also use audio data captured during the surgical procedure. The audio data can include sounds emitted by the surgical instrumentation system 106 while activating one or more surgical instruments 108. Alternatively, or in addition, the audio data can include voice commands, snippets, or dialog from one or more actors 112. The audio data can further include sounds made by the surgical instruments 108 during their use.

In one or more examples, the machine learning models can detect surgical actions, surgical phases, anatomical structures, surgical instruments, and various other features from the data associated with a surgical procedure. The detection can be performed in real-time in some examples. Alternatively, or in addition, the computing system 102 analyzes the surgical data, i.e., the various types of data captured during the surgical procedure, in an offline manner (e.g., post-surgery).

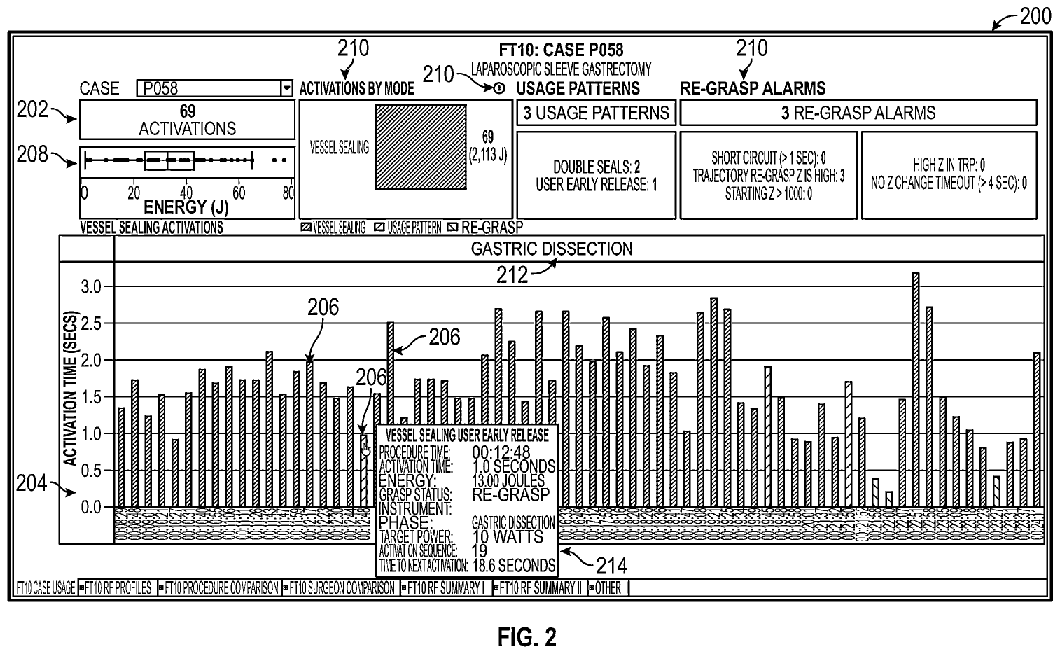

FIG. 2 depicts an example report of an analysis performed by the computing system 102 using the surgical data. While some of the examples and drawings herein are provided regarding “activations” of instruments 108, it should be understood that in other aspects of the technical solutions herein, features described herein are applicable to other types of usage of the instruments 108. In some aspects, separate reports for different types of usages are generated. In other aspects, a common report for a combination of different types of usages is generated. The report 200 is user-interactive. The report 200 can be displayed via the user interface of the computing system 102. Alternatively, or in addition, the report 200 is displayed via another device (not shown) that is in communication with the computing system 102. The report or parts thereof can be added to an electronic medical record of a patient in one or more aspects. The report 200 can be for the entire surgical procedure or a portion of the surgical procedure. FIG. 2 depicts an example of a portion of the surgical procedure, for example, a particular phase of the surgical procedure. In one or more examples, the report 200 is displayed for a phase that is automatically detected by the machine learning models of the computing system 102.

The report 200 can include a user-informative element 202 that indicates a number of activations during the phase(s) of the surgical procedure associated with the report 200. Further, the report 200 includes a timeline 204 that includes a user-interactive element 206 representing each of the activations performed. The timeline 204 indicates timestamps at which the activation was initiated. Further, the timeline 204 indicates a duration of each activation. The duration can be depicted using a visual attribute of the user-interactive element 206, for example, length, width, color, transparency, border, etc.

Additionally, the report 200 includes a user-informative element 208 that indicates an amount of energy applied during the phase(s) of the surgical procedure associated with the report 200. Further, the report 1500 includes the timeline 204 that includes a user-interactive element 206 representing each of the activations performed.

Based on the energy applied for each activation and/or the duration of the activation, a type of the activation can be determined by the machine learning model. In one or more examples, the type of the activation is indicated using a visual attribute of the user-interactive element 206, for example, color, transparency, border, etc. For example, in FIG. 2 vessel sealing type activations (blue), double sealing type activations (teal), and re-grasps type activations (orange) are shown using different colors. It is understood that other types of activations can be detected, and that different visual attributes can be used to represent the types of activations in other examples. In one or more examples, the visual attributes used to depict various detections by the machine learning model are user configurable.

Further yet, the report 200 includes user-informative elements 210 for each type of activation detected. In one or more examples, if the types of activations can be further subclassified, the user-informative elements 210 include details, such as a number of the sub-types of activations, thresholds associated with such sub-types, etc.

Although not shown, the report 200 can include additional metrics, parameters, or features, such as those listed in the following table:

| Parameter/Feature | Calculation Details |
|---|---|
| Case Duration | Time between “Patient In” annotation and “Patient Out” |
| | annotation. |
| Patient In to Cut | Time between “Patient In” annotation and start of video |
| | recording/“Operation Start” annotation. |
| Operating Time/Cut to Close | Time between start of video recording/“Operation Start” |
| | annotation and “Operation End” annotation. |
| Close to Patient Out | Time between “Operation End” annotation and “Patient |
| | Out” annotation. |
| Camera Out % | Summation of Camera Out periods divided by the |
| | Operating Time × 100. |
| Efficiency and Safety | Benchmarks for “Component” and “Proficient” defined |
| Performance Benchmarking | using Performance metric distributions of clinical data |
| | collected. |
| Maximum Error Level | Defined using Error Tracking metric distributions of |
| | clinical data collected. |
| Proficiency Measure (Safety) % | Number of Safety Performance metrics user has reached |
| | the “Proficient” benchmark for divided by the total |
| | number of metrics × 100. |
| Proficiency Measure (Efficiency) | Number of Efficiency Performance metrics user has |
| % | reached the “Proficient” benchmark for divided by the |
| | total number of metrics × 100. |
| Case Duration Outlier | Outlier detection analysis calculation for case durations |
| | using a Boxplot Measure suitable for skewed |
| | distributions. |
| Phase Duration Outlier | Outlier detection analysis calculation for phase durations |
| | using a Boxplot measure suitable for skewed distributions. |
| Instrument Standardization | A measure between 0 (instruments used different in every |
| | case) and 1 (instruments used are identical in every case). |
| Unused Pref Card Instruments | Number of instruments unused in case but included in |
| | Preference Card (represents potential cost wastage- |
| | instruments opened/requiring sterilization). |
| Used Pref Card Instruments | Number of instruments used in case and included in |
| | Preference Card. |
| Used Instruments not on Pref. | Number of instruments used in case but not included in |
| Card | Preference Card (represents potential time wastage- |
| | instruments not present in operating theatre but required). |
| Workflow Standardization | A measure between 0 (phase workflow different in every |
| | case) and 1 (phase workflow the same in every case). |
| Variation over Surgery | A measure of variation (entropy) across a set of aligned |
| | surgical workflows. |
| Expected Case Values | Expected case value ranges (e.g. case duration) can be |
| | predicted using factors to identify similar cases (e.g., |
| | patient factors). |
| Significant Workflow Deviation | Indicates if a case workflow significantly deviates from a |
| Indicator (Workflow Variation | surgeon's usual workflow. |
| tag) | |
| Significant Instrument Deviation | Indicates if a case instrument list significantly deviates |
| Indicator (Instrument Variation | from a surgeon's usual instrument list. |
| tag) | |
| Patient Outlier | Indicates if a patient's characteristics significant deviate |
| | from the usual patient characteristics. Requires a |
| | multivariate outlier detection algorithm. |
| Case Complexity/Patient Factor | Method to remove the effect of case factors to allow for |
| Effect Elimination | comparable data analytics such as Propensity Score |
| | Matching, Stratification, Regression Adjustment etc. |
| Learning Curve Prediction | Method to estimate a confidence interval for learning |
| | curve prediction. |
| Duration System Time On (s)- | Duration between the event when the Start Surgery button |
| Learning Curve | is pressed on ORTI Display and the event when the |
| | System Power button is used to deactivate system either |
| | on Tower or armrest of Surgeon Console. |
| Duration First Trocar Attach to | Duration between the event when the first trocar is |
| Last Trocar Detach (s)-Learning | attached to the robot arm and the event when the last |
| Curve | trocar is detached from the robot arm (and no instruments |
| | are attached). |
| Duration Active Console Time | Total duration when either of two Surgeon Hand |
| (s)-Learning Curve | Controllers (AKA Control Devices) are in unlocked |
| | mode. |
| Duration when surgeon is | Total duration when 1) the surgeon is inattentive (i.e. head |
| inattentive and a minimum of one | tracking system determines that surgeon is not looking at |
| Surgeon Hand Controller (AKA | the console) AND 2) at least one Surgeon hand controller |
| control device) is in unlocked | is unlocked. The case where the surgeon is Inattentive and |
| mode (s)-Novice Error | both Surgeon Hand Controllers are locked does not count. |
| Number of Instrument | A continuous curvilinear movement of the First Proximal |
| translational movements | Joint (FPJ) of the instrument of 3 mm or more counts as a |
| Learning Curve | movement. Changing the movement direction by 90 |
| | degrees for a continuous curvilinear movement of at least |
| | 3 mm, counts as a new movement. This will be measured |
| | for each instrument that is in use. Instrument wristed |
| | motion and jaw open/close motions are not considered as |
| | instrument movement. |
| Instrument translational path | Total distance travelled by instrument (Based on FPJ |
| length (mm)-Learning Curve | movement that is tracked when surgeon is attentive and at |
| | least one Surgeon Hand Controller is unlocked. This does |
| | NOT include instrument wrist or jaw movement). This |
| | will be measured for each instrument that is in use. |
| Instrument translational velocity | The velocity with which the FPJ of the instrument moves. |
| (mm/s)-Learning Curve | This will be measured for each instrument that is in use. |
| Primary Instrument idle time | Duration of time when primary instrument is idle and |
| while unlocked and attentive (s)- | when system is in a mode where surgeon is looking at the |
| Novice Error | monitor, at least one Surgeon hand controller is unlocked, |
| | and the surgeon is not moving the instruments. Instrument |
| | is defined to be idle if it is not in movement. This will be |
| | measured for each instrument that is in use. |
| Number of Curvilinear Camera | Movement is defined as curvilinear movement of the tip |
| movements-Learning Curve | of the endoscope. A continuous curvilinear movement of |
| | 3 mm or more counts as a movement. Changing the |
| | movement direction by 90 degrees for a continuous |
| | curvilinear movement of at least 3 mm, counts as a new |
| | movement. |
| Number of camera “roll” | The number of times when a continuous roll of endoscope |
| movements-Learning Curve | occurs above a threshold limit (to be defined). Roll is |
| | distinct from curvilinear movement. |
| Camera path length (mm/s)- | Total curvilinear distance travelled by tip of endoscope |
| Learning Curve | when surgeon is looking at the monitor and at least one |
| | surgeon hand controller is unlocked. This will be |
| | measured for each endoscope that is used. |
| Clutch usage during position | Total number of occurrences when any clutch is engaged |
| control-Learning Curve | (foot pedal or left hand trigger or right hand trigger on |
| | Surgeon Hand Controller) while system is in position |
| | control (Surgeon looking at the monitor and at least one |
| | surgeon hand controller is unlocked). This does not |
| | include attentive-inattentive transitions from head |
| | tracking. |
| Fourth Arm Swap Failure Rate | Number of events when the fourth arm is successfully |
| (Novice Error) | swapped using either screen based GUI or foot pedal |
| | divided by the number of events when the fourth arm is |
| | attempted to be swapped using either screen based GUI or |
| | foot pedal. |
| Energy Bank Activation Failure | Number of events when energy bank was successfully |
| Rate (Novice Error) | activated divided by the number of events when energy |
| | bank was attempted to be activated-this includes all |
| | energy activations (monopolar-cut, coag; bipolar; |
| | ligasure-cut, seal). |
| Energy Activation Failure Rate | Number of events when energy was successfully activated |
| (Novice Error) | divided by the number of events when energy was |
| | attempted to be activated-this includes all energy |
| | activations (monopolar-cut, coag; bipolar; ligasure-cut, |
| | seal). |
| Number of calibrated Instrument- | Number of detachments of a calibrated Instrument |
| instrument exchanges- | followed by an attachment of a instrument with a different |
| Learning Curve | serial number to Instrument drive unit (IDU), for every |
| | arm that is in use. |
| Number of endoscope-endoscope | Number of events a different endoscope is plugged into |
| exchanges-Learning Curve | the Stortz box. |
| Number of endoscope-instrument | Number of detachments of an endoscope followed by an |
| exchanges-Learning Curve | attachment of a instrument or vice versa to IDU, for every |
| | arm that is in use. |
| Hand controller workspace range | The larger of the two radii of motion of the user's |
| (cm)-Learning Curve | working volume on left and right hand Surgeon Hand |
| | Controllers. |
| Workspace limit of Robot arm/ | Number of times workspace limit reached on robot arm/ |
| instrument-Novice Error | instruments. |
| WorkSpace Limit of Surgeon | Number of times workspace limit reached on either or |
| Hand controllers-Novice Error | both Surgeon Hand Controllers. |

The report 200 is user-interactive. In one or more examples, the user (e.g., actor 112) can select the phase of the surgical procedure for which the visual information is generated and depicted. For example, a user-interactive selector 212 facilitates the user to change the phase that is being analyzed and visualized. Alternatively, or in addition, the user can view the activations during a particular timeframe of the surgical procedure by altering the timestamps shown on the timeline 204.

Further, each of the user-interactive element 206 that represents an activation, in response to a first interaction, such as a hover, a click, a right-click, a touch, a voice command, etc., provides detailed information about that activation via a user-informative element 214. For example, the user-informative element 214 can identify the procedure being performed, a phase of the procedure, the activation time, the activation duration, the amount of energy supplied, the grasp status (if applicable), a prescribed (expected) amount of power for the activation, a activation sequence number, and a duration between this activation and a subsequent activation, among other information.

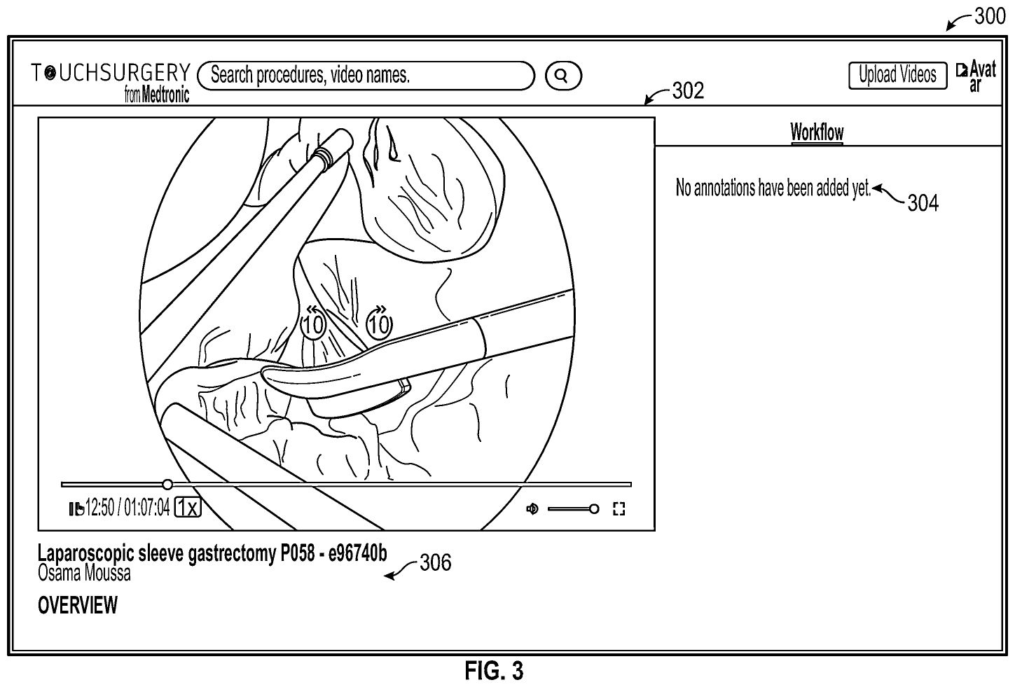

Further yet, in response to another interaction with the user-interactive element 206, e.g., click, double click, right click, etc., the visual report 200 displays a view 300. FIG. 3 depicts an example view 300. The view 300 includes a video playback 302 of the portion of the surgical procedure corresponding to the activation associated with the interacted user-interactive element 206. The video playback 302 can include a portion of the video stream that is recorded from the endoscopic view, from the external view (outside the patient), or a combination of both the endoscopic and the external views. The video playback 302 can also be interactive where the user can rewind, forward, change playback speed, etc.

In one or more examples, the user can view/add annotations 304 to the portion of the video associated with the selected activation. The view 300 can further include additional details about the activation, such as those in the user-informative element 214, or any different details.

FIG. 4 depicts another example of the report 200. In addition to the information that is visually depicted in the example in FIG. 2, the report 200 of FIG. 4 further categorizes the user-interactive elements 206 according to the surgical actions for which the activations are performed. The surgical actions, such as angle of his dissection, part removal, etc. are annotated and the corresponding actions for such actions are marked using a user-indication 220. The user indication 220 can be a line, a bounding box, a color, a border, or any such visual attribute. The report 200 in FIG. 4 also depicts different types of activations that may be detected.

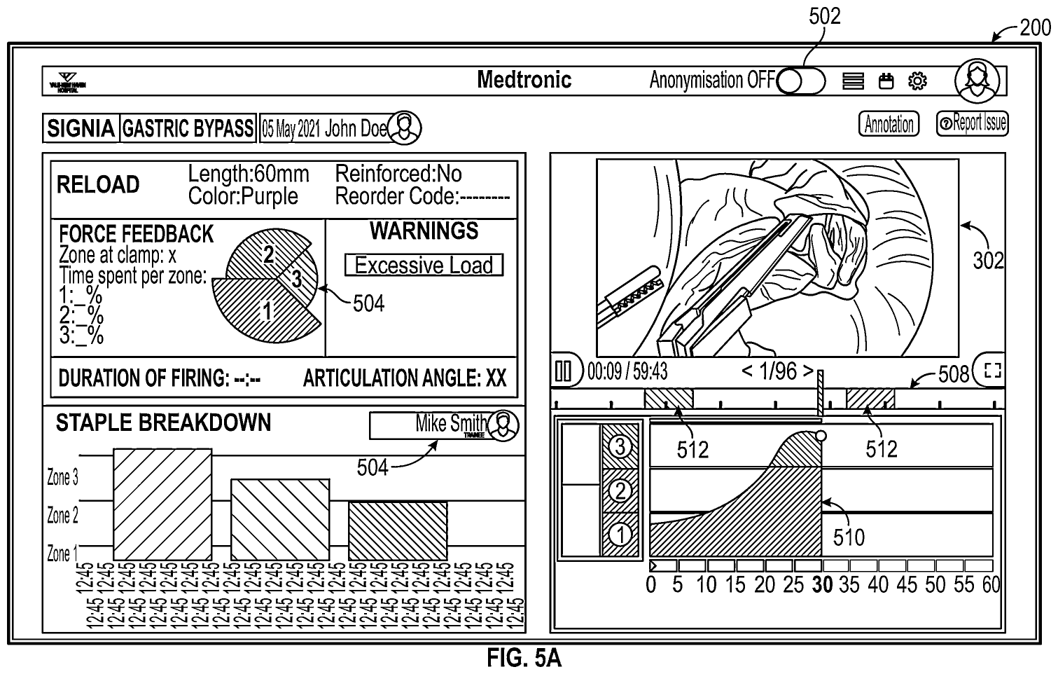

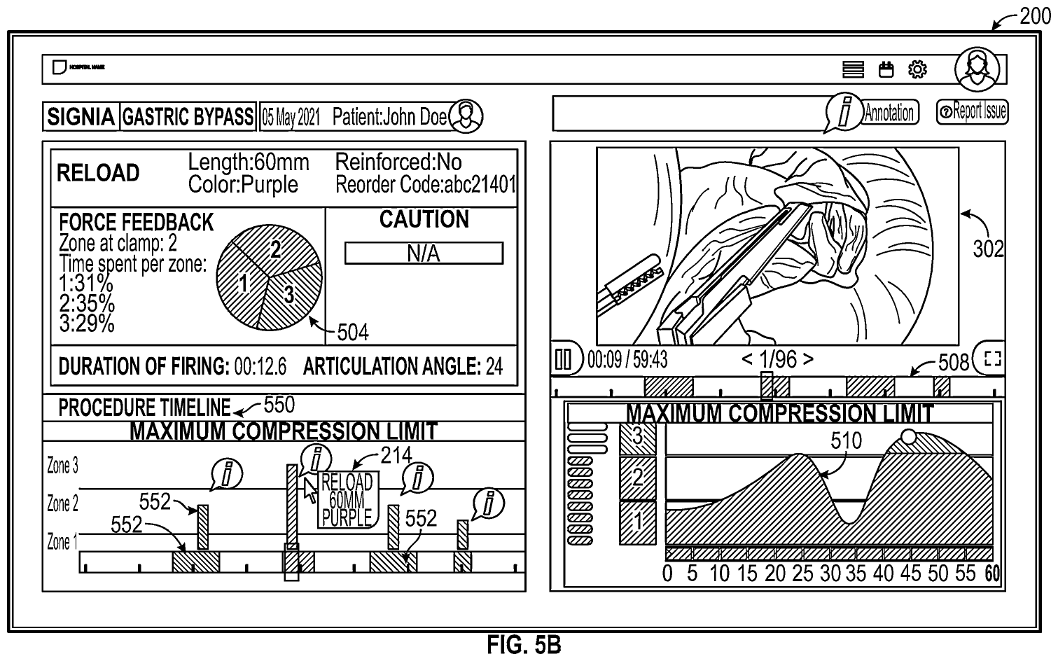

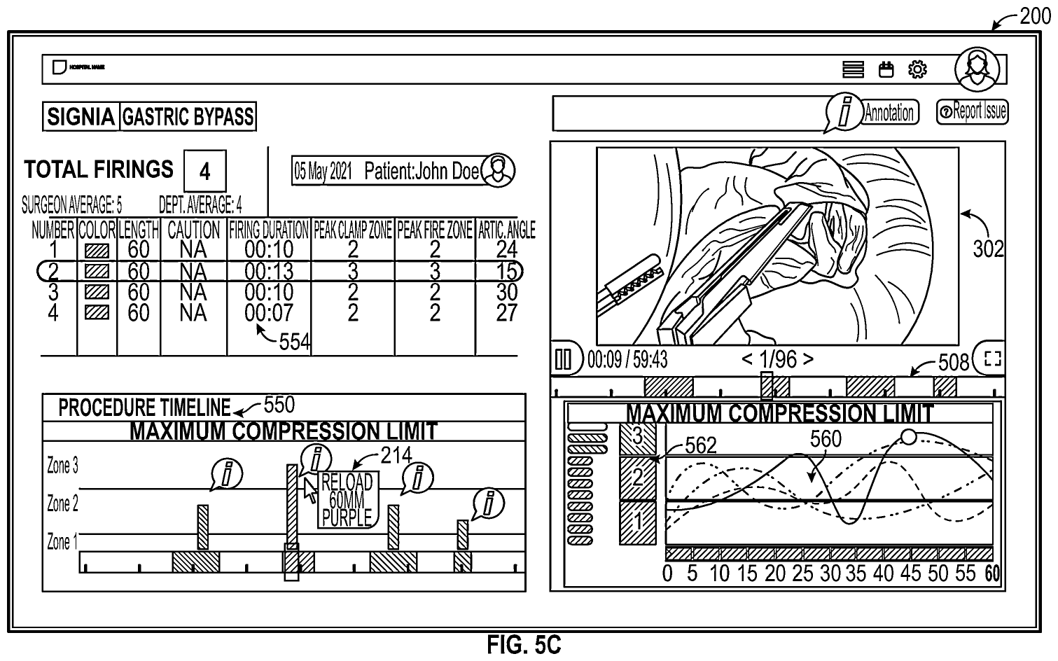

In one or more examples, the report 200 can be configured by the user to display the information using different elements. FIG. 5A depicts another example user-interactive report 200. In one or more examples, the user can select via an anonymization selector 502 whether the information being displayed should be anonymized. It is understood that the selector 502 can be a different type of user-interactive element from the one used in FIG. 5A. For example, with anonymization switched off, the report 200 indicates the name of the actor 112 that performed one or more surgical actions (504). Further, the user can select, instead of the timeline (204), to display a different type of chart 506 that indicates a time spent per zone for a particular type of surgical action.

Additionally, in the report 200 of FIG. 5A, the user can select the video playback 302 to be a constant part of the view. The video playback 302 can be associated with an interactive-playback selector 508. The interactive-playback selector 508 includes visual depictions 512 of phases, surgical actions, and other such events along a timeline of playback of the captured video from the surgical procedure. The user can select to playback a portion of the video corresponding to a particular phase, surgical action, etc., by selecting the visual depiction 512, for example, by clicking, double clicking, etc.

In addition, the interactive-playback selector 508 displays a chart 510 that indicates the activations performed at each timepoint in the surgical procedure as the video is played back. The chart 510 indicates the activation initiation, duration, energy applied at the activation, and other such information. In one or more examples, the chart 510 can be replaced by the timeline 204.

Other setups of the report 200 are possible according to the user's preferences.

FIG. 5B depicts another example of user-interactive report 200. In addition to several user-interactive elements/modules/widgets that are described herein, the report 200 includes a procedure event timeline 550 that depicts events 552 of particular types such as, firing a stapler, incision, clamping, etc. performed using one or more of the surgical instruments. In one or more aspects, the events 552 are represented using a visual attribute (e.g., color, shape, shading, character, etc.) to distinguish the type of the event. For example, a different color can be used for each respective event type, e.g., blue for stapling, green for incising, yellow for clamping, etc. Alternatively, or in addition, the color can represent different types of staples used, e.g., magenta for staple-type 1, cyan for staple-type 2, etc.

The same visual attribute (e.g., color) can be used to depict the event in the playback timeline 508. Accordingly, when a user interacts with either the playback timeline 508 or the procedure event timeline 550, the other timeline (508/550) is altered/manipulated in conjunction. Further, another one of the visual attributes of the events 552 can be used to depict information associated with the event, for example, an amount of pressure/compression applied when performing the event 552 (e.g., clamping) can be depicted by the length of a bar representing the event 552. The events 552 in the procedure event timeline 550 can be highlighted when the corresponding event is displayed during the video playback 302.

FIG. 5C depicts another example of user-interactive report 200. In addition to several user-interactive elements/modules/widgets that are described herein, the report 200 includes a list of events 554 performed during the surgical procedure. In some aspects the list of events 554 is a list of specific type of events, such as firing of a stapler. It is understood that other types of events can be populated in the list of events 554 in other aspects. The list of events 554 shown in FIG. 5C includes one or more factors associated with the events. For example, in the case firing of staples, length (i.e., duration) of the event, peak clamp zone, peak fire zone, articulation angle, etc. can be listed for each event.

Additionally, the report 200 can include a graphical comparison 560 of the events in the list of events 554. The graphical comparison 560 can visually depict each of the events. For example, in the case of the firing of staples, each firing is shown as a line graph showing an amount of compression applied as each event was performed. The graphical comparison 560, in some aspects, is accompanied by a zone visualizer 562. The zone visualizer 562 indicates a category (i.e., zone) of the amount of compression applied when firing the staple in the case of FIG. 5C. It is understood that the zone visualizer 562 can be dynamically adjusted based on the type of events being compared by the graphical comparison 560. In one or more aspects, the zone visualizer uses a visual attribute, for example, color, to depict when the amount of compression applied is above a certain threshold (e.g., zone 3), or within a predetermined range of threshold (e.g., zone 1, zone 2, zone 3, etc.). The graphical comparison 560 can use different colors, or other visual attributes, to distinguish between the different events from the list of events 554.

In one or more aspects, the list of events 554 is user interactive. A user can select an event from the list of events 554, and in response, the video playback 302 can display a portion of the video of the surgical procedure when the selected event is being performed.

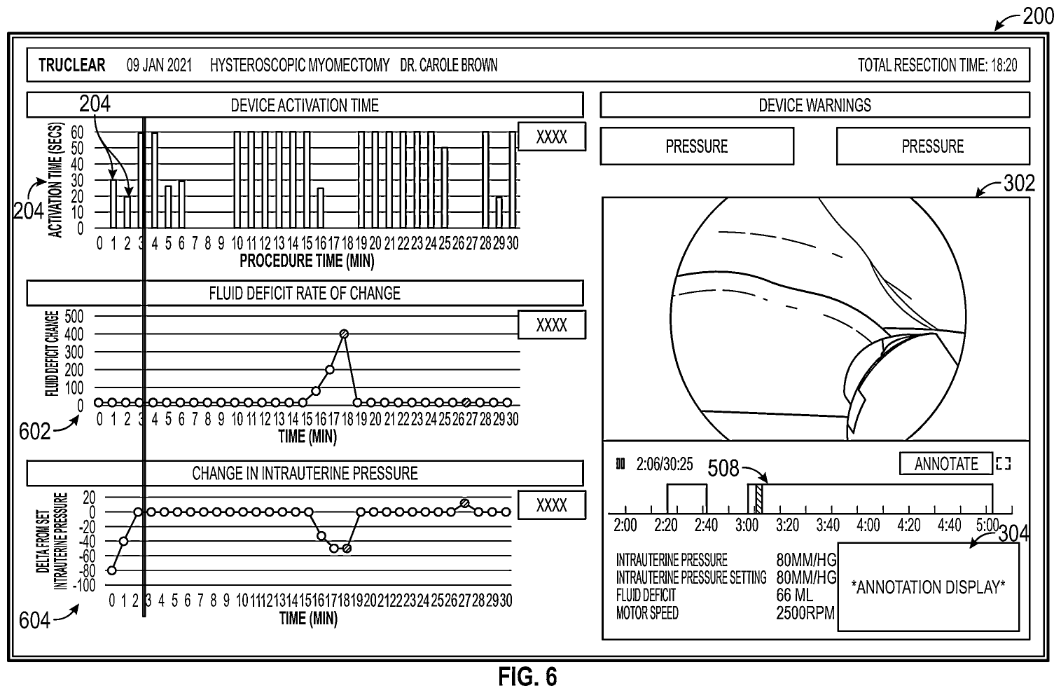

FIG. 6 depicts another user-interactive report 200 according to one or more examples. Here, the timeline 204 with the user-interactive elements 206, the video playback 302, and the interactive-playback selector 508 are included in the report 200. In addition, the user is provided the option to add an annotation 304 to an activation and/or a timepoint in the video playback 302. In one or more examples, the timeline 204, the video playback 302, and the interactive-playback selector 508 work in conjunction. For example, if the user selects a user-interactive element 206 from the timeline 204, the interactive-playback selector 508 advances (or reverses) to the corresponding timepoint, and the video playback 302 displays the corresponding portion of the video of the surgical procedure. In addition, the report 200 of FIG. 6 includes an interactive chart 602 of a fluid deficit rate of change and a chart 604 of a change in intrauterine pressure. Such charts 602, 604, are based on one or more sensor measurements indicating physical measurements from the patient 110. It is understood that other types of measurements can be included in the charts 602, 604, and/or additional charts for other measurements can be included in the report 200.

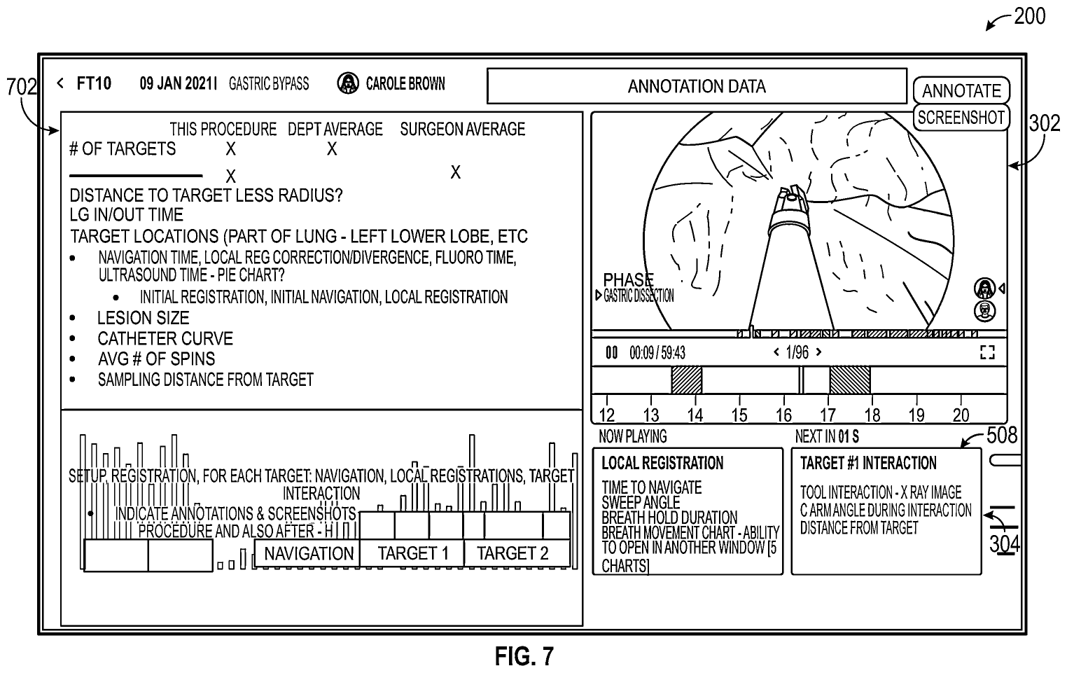

FIG. 7 depicts yet another user-interactive report 200 according to one or more examples. In this case, a user-informative element 702 is included. The user-informative element 702 includes information that displays one or more statistics from the surgical procedure that is presently being analyzed in comparison with baseline, threshold, or standardized statistics. For example, a number of targets met during the present surgical procedure in comparison to an average number of targets met by actors 112 from a department, or the same actor's average are shown. Other types of such statistics that are recorded throughout the surgical procedure can be also shown in other examples. Such information can be used to train new medical personnel, for example, by identifying phases, surgical actions, or other types of events during the surgical procedure where improvement can be made.

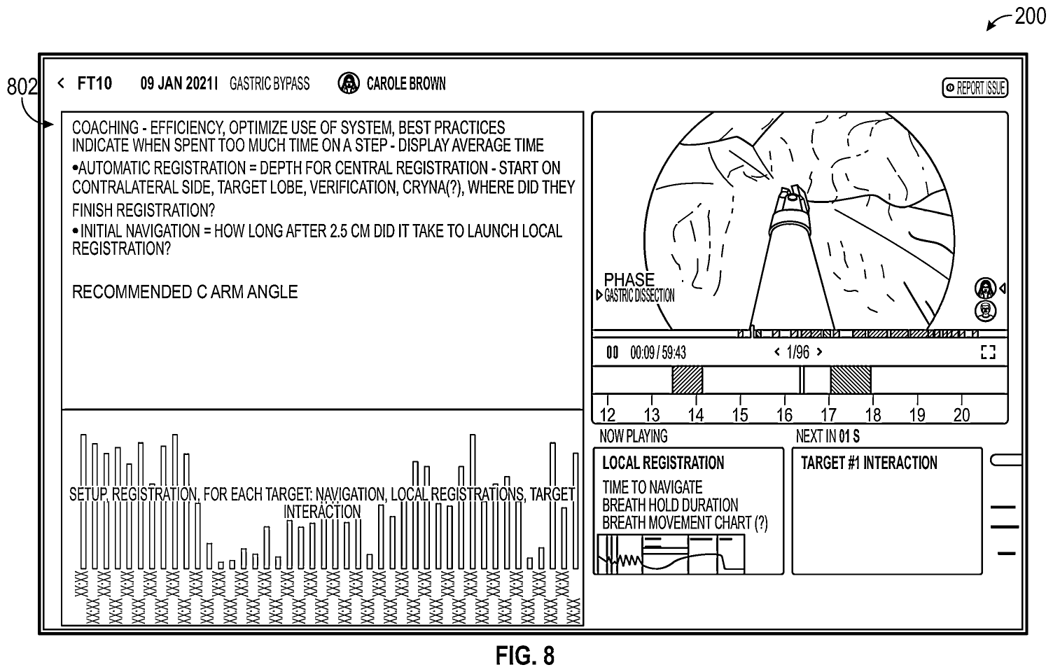

As shown in FIG. 8, the report 200 can further include a user-informative element 802 that displays one or more suggestions for the actor to improve his/her statistics when performing the surgical procedure. For example, the user-informative element 802 can indicate changes in angles when using particular surgical instruments 108, changes in activation durations, and other such changes to improve the statistics, and in turn the performance/outcome of the surgical procedure.

The computing system 102 can further facilitate comparing and training statistics from one surgical procedure with one or more other surgical procedures, and depict the comparison visually in an interactive report. Such reports can be used to train and improve performance of one or more actors 112. The reports can, in turn, improve the performance and outcomes of the surgical procedures.

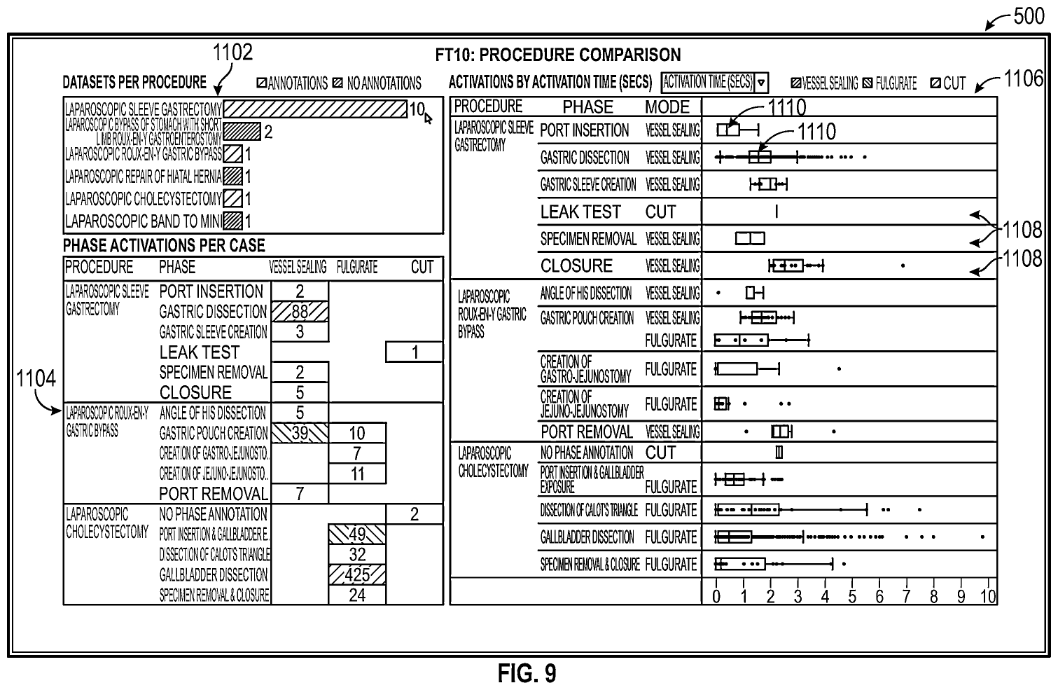

FIG. 9 depicts an example report 500 of a comparison of surgical procedures. The report 500 facilitates analyzing multiple surgical procedures at a time, as opposed to a single surgical procedure as was the case with reports 200, 400.

In some examples, surgical procedures of the same type are compared in the report 500. In one or more examples, different types of surgical procedures for which the surgical data is available are shown in a user-informative element 1102. The different types of surgical procedures can be further categorized based on an attribute of the corresponding surgical data. In the example of FIG. 9, the data is categorized based on whether it has been annotated. In one or more examples, the user can select a particular type of surgical procedure from the element 1102 to interactively change the information in other elements of the report 500.

The report 500 can include a user-informative element 1104 that indicates activations in each phase for the surgical procedures being analyzed. A table can be generated and displayed that shows information for the different types of activations that are performed in different phases of each of the surgical procedures. The activations can be depicted using different visual attributes, and the information displayed can include a number of such activations.

Further, a user-informative element 1106 can depict additional details including timelines 1108 for each activation. The timelines 1108 represent the time when the activation was initiated, and a duration of the activation using a dimension (e.g., length) of the user-interactive element 1110 used to represent each activation. Additionally, in some examples, the user-interactive element 1110 also depicts an energy supplied for the activation using another dimension (e.g., height).

The computing system 102 can further facilitate comparing and training statistics based on different actors 112, for example, surgeons, and depict the comparison visually in an interactive report. Such reports can be used to train and improve performance of one or more actors 112. The reports can, in turn, improve the performance and outcomes of the surgical procedures. Further, such reports can facilitate identifying one or more actors 112 that are performing an action, phase, or surgical procedure better in relation to others, so that their protocols may be replicated for improving the performance of the other actors 112.

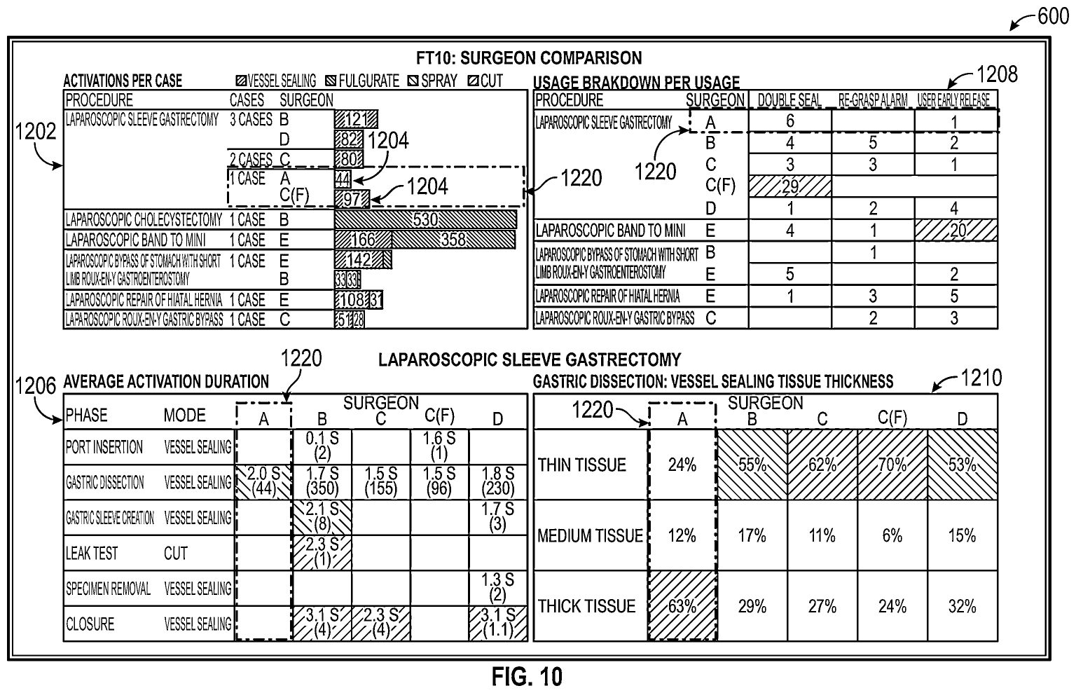

FIG. 10 depicts an example report 600 that visually depicts surgical data across different surgeons. It is understood that in other examples, different types of actors 112 can be used, such as nurses. The report 600 includes a user-informative element 1202 that indicates a number of activations per surgical procedure performed by the different surgeons. The number of activations can be represented by a bar chart 1204. Further, the visual attributes of the bar chart 1204 can be configured to represent different types of activations.

A user-informative element 1206 depicting an average activation duration is also included in the report 600. The phases in which the activations are performed can also be depicted in the user-informative element 1206. Another user-informative element 1208 indicates the types of activations performed by each surgeon during each different type of surgical procedures. Yet another user-informative element 1210 can represent proportions of tissue thickness for each surgeon when performing a particular surgical action.

In one or more examples, the user can select a particular surgeon in any of the user-informative elements 1202, 1206, 1208, 1210, and the data associated with the selected surgeon is highlighted (or marked) in each of the user-informative elements of the report 600. For example, the highlighting can include a graphical overlay 1220. However, it is understood that any other type of highlighting can be performed.

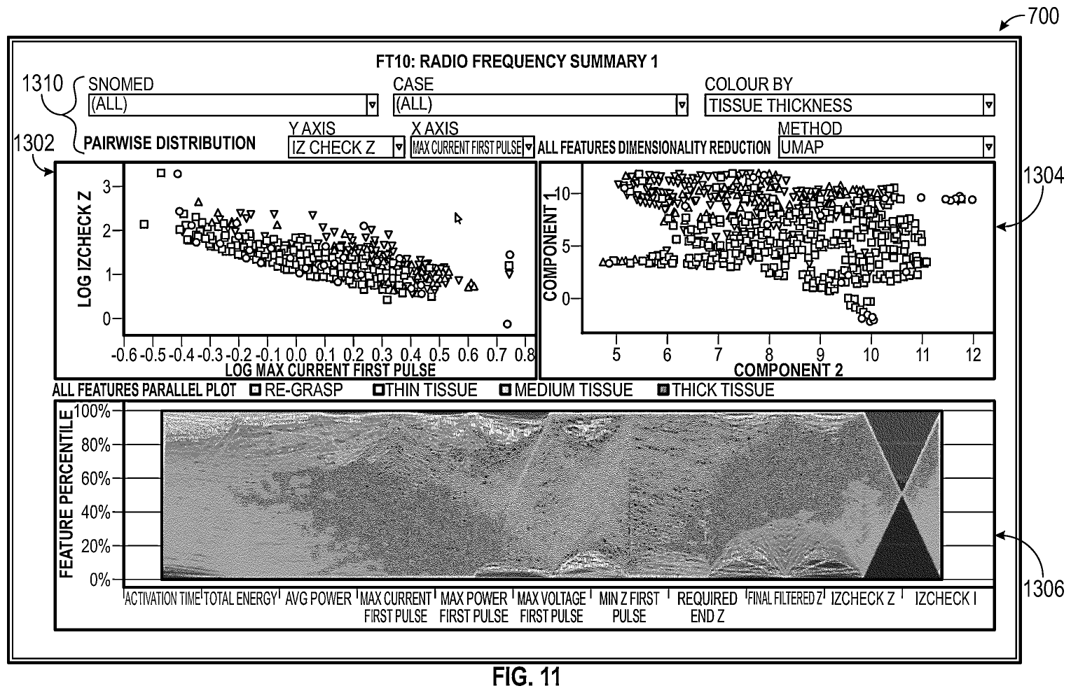

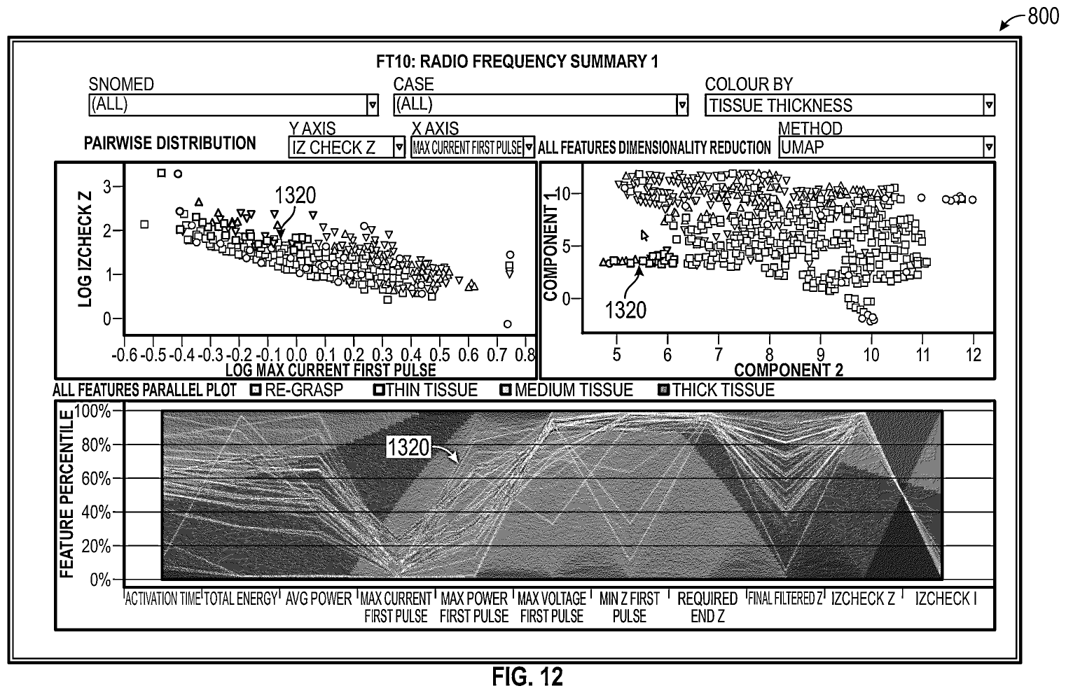

FIG. 11 depicts an example report 700 that displays various user-interactive charts 1302, 1304, 1306. The information displayed in the charts 1302, 1304, 1306 can be configured using the selectors 1310. The selectors 1310 can facilitate a user to select what attribute is charted along a particular axis (X, Y) in the charts 1302, 1304, 1306. Additionally, the selectors 1310 facilitate selecting the visual attributes of the information that is displayed on the charts 1302, 1304, 1306. For example, the visual attributes such as color, shape, dimensions, borders, etc. can be modified based on type of surgical procedure, type of activation, amount of energy applied, or any other such attribute.

The charts 1302, 1304, 1306 include user-interactive elements 1320 representing each activation. The charts 1302, 1304, 1306 work in a coordinated manner. For example, when one or more user-interactive elements 1320 are selected in one of the charts 1302, 1304, 1306, the user-interactive elements corresponding to the activations of the selection are highlighted in the remaining charts 1302, 1304, 1306. Further user interaction (e.g., click, double click, etc.) with the selected user-interactive elements 1320 (on any of the charts 1302, 1304, 1306), can navigate the user to other reports, such as the view 300 to provide the video playback 320 of the corresponding activation.

Examples described herein facilitate providing a user-interactive system to visualize and analyze large amounts of data associated with the CAS system 100. Generating such user-interactive reports of the large amounts of data is not practical for a human, and hence, the technical solutions described herein provide a practical application to address technical challenges and provide improvements to CAS systems. For example, the technical solutions described herein facilitate service providers to review surgical procedures performed using the CAS system over a certain period of time (e.g., month, quarter, etc.) and provide feedback to the hospital, actors, or any other stake-holder. Further, the technical solutions described herein facilitate troubleshooting and diagnosing complaints about the CAS system. Additionally, the technical solutions described herein facilitate training actors that perform surgical procedures using the CAS systems, in turn helping to improve the performance and outcomes of the surgical procedures.

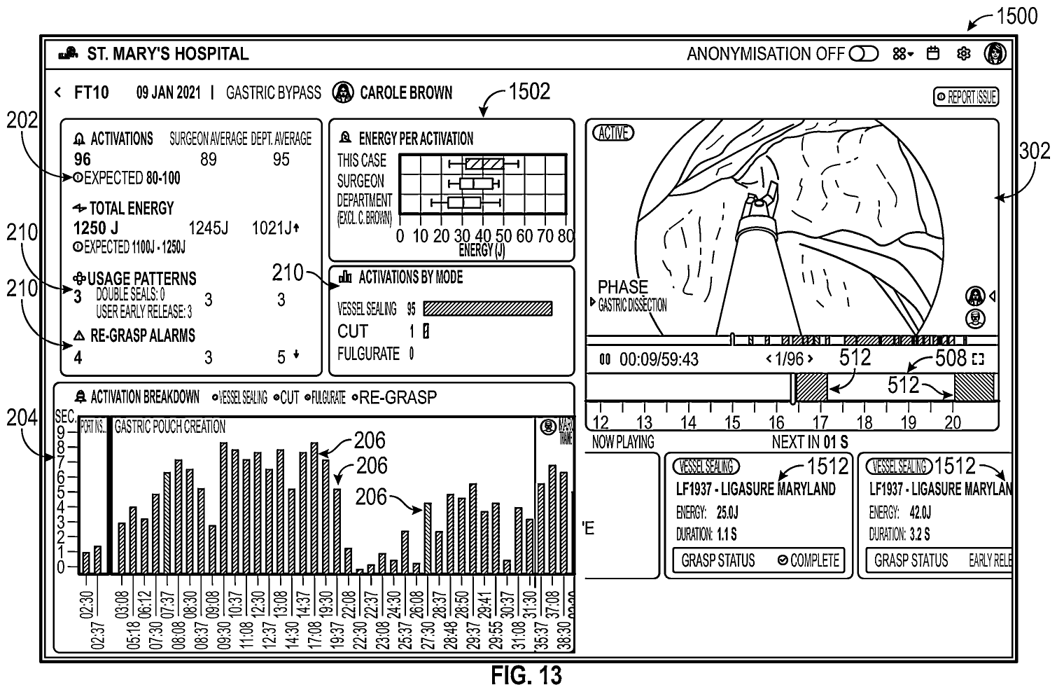

FIG. 13 depicts an example report 1500 of an analysis performed by the computing system 102 using the surgical data. The report 1500 is user-interactive. The report 1500 can be displayed via the user interface of the computing system 102. Alternatively, or in addition, the report 1500 is displayed via another device (not shown) that is in communication with the computing system 102. The report 1500 can be for the entire surgical procedure or a portion of the surgical procedure. FIG. 13 depicts an example of a portion of the surgical procedure, for example, a particular phase of the surgical procedure. In one or more examples, the report 1500 is displayed for a phase that is automatically detected by the machine learning models of the computing system 102. The report 1500 includes the user-informative element 202 that indicates a number of activations during the phase(s) of the surgical procedure.

Additionally, the report 1500 includes video playback 302 of the portion of the surgical procedure corresponding to the activation associated with the interacted user-interactive element 206. Alternatively, the video playback 302 can display a video based on some other user-interaction with the report 1500. For example, the user can initiate playback of the entire surgical procedure. Alternatively, or in addition, the user can interact with other user-interactive elements of the report 1500 to trigger a corresponding portion of the video to be selected and played back. In one or more examples, the user can view/add annotations 304 to the portion of the video associated with the selected activation. The view 300 can further include additional details about the activation, such as those in the user-informative element 214, or any different details.

The video playback 302 can be associated with an interactive-playback selector 508. The interactive-playback selector 508 includes visual depictions 512 of phases, surgical actions, and other such events along a timeline of playback of the captured video from the surgical procedure. The user can select to playback a portion of the video corresponding to a particular phase, surgical action, etc. by selecting the visual depiction 512, for example, by clicking, double clicking, etc.

The visual attributes of the elements 206 that are displayed on the timeline 204 are selected to display the one or more visual depictions. Further, in some aspects, the report 1500 includes information elements 1502 that are populated to provide a comparison of one or more performance of one or more actions in the surgical procedure with other surgical procedures. The user can select what details are to be compared and presented in the elements 1502. For example, the user can select to compare energy per activations during this particular surgical procedure with other surgical procedures (of the same type) performed by the same surgeon. Alternatively, or in addition, the energy per activations can be compared with other surgeons in the same department (or hospital/institute). It should be understood that other types of information can be compared in other aspects.

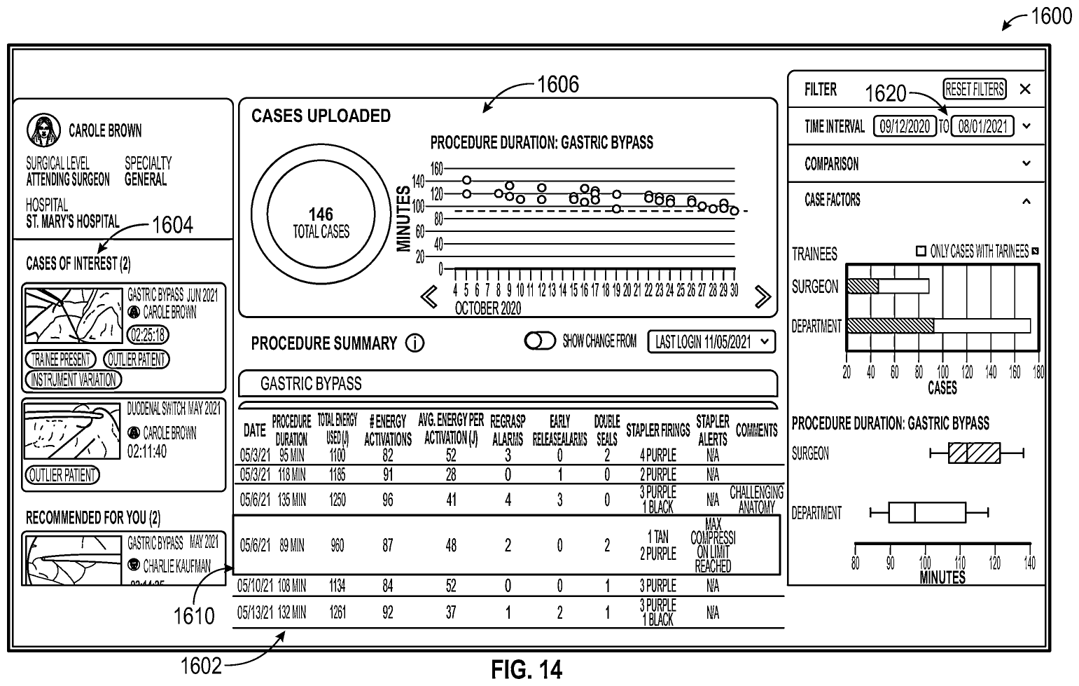

FIG. 14 depicts a user-interactive summary report 1600 for analysis of multiple surgical procedures performed according to one or more aspects. The report 1600 can facilitate a user to filter which surgical procedures are to be included in the report 1600 via a user interactive element 1620. Surgical procedures can be filtered using several factors such as when performed (e.g., date range), duration (i.e., length of procedure), case factors (e.g., performed by particular surgeon, trainee; performed at a particular hospital; performed using a particular system 102; etc.), etc.

The selected surgical procedures can be displayed, for example, as a list, a table, or any other such format by a user-interactive element 1604. Various details of the surgical procedures can be listed in the user-interactive element 1604. Annotations added by one or more medical personnel during the surgical procedure can also be included in the displayed information.

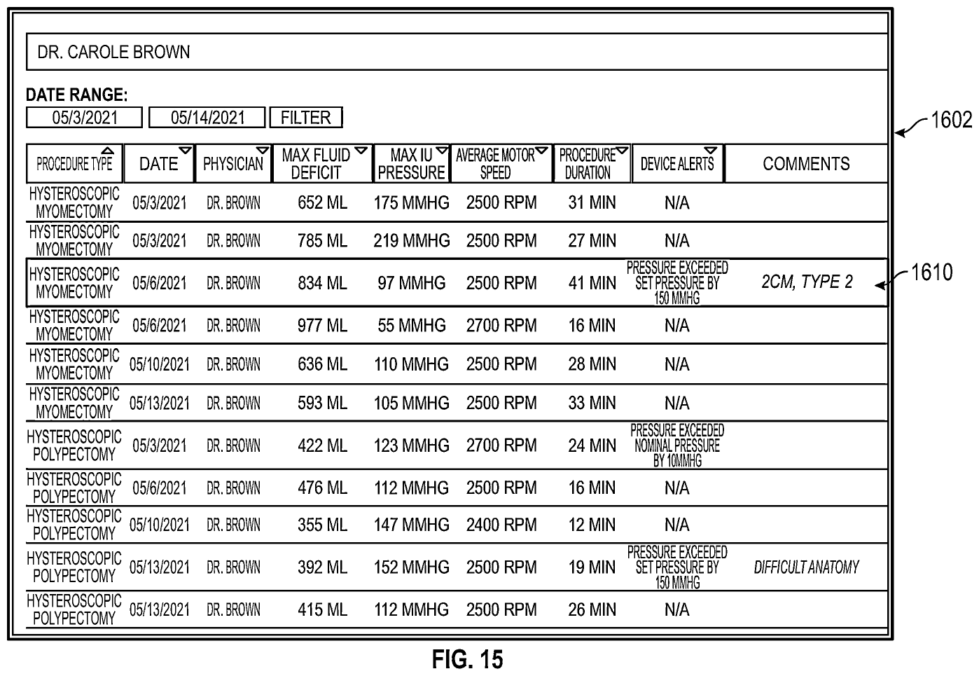

FIG. 15 depicts another view of the list of surgical procedures 1602. Here only the surgical procedures of a specific type performed by a specific surgeon within a specific time range are listed. Several parameters/attributes/factors associated with the surgical procedures are listed/tabulated. In some aspects, the attributes to be listed/tabulated can be selected by the user. It is understood that in other aspects, the surgical procedures can be filtered based on other attributes.

In addition, based on an analysis of the selected surgical procedures 1602, the report 1600 is populated with a user-interactive element for cases of interest 1604. The cases of interest 1604 can include surgical procedures that the same surgeon had performed earlier with factors common to those in the selected surgical procedures. Alternatively, or in addition, the cases of interest 1604 include surgical procedures performed by other surgeons with one or more common factors as those in the selected surgical procedures. The cases of interest 1604 can further include portions of video of the surgical procedures that a user can playback.

In some aspects, a user-interactive element 1606 displays one or more graphics to summarize the surgical procedures. For example, the summarization can include representing the surgical procedures on the one or more graphical visualizations based on one or more factors. For example, a duration of the surgical procedure can be used to categorize the surgical procedures. Any other factor, or a combination of factors, can be used to categorize the surgical procedures.

The user can select an entry 1610 from the list of surgical procedures 1602, for example, by a click, a touch, a voice input, etc. The selected entry 1610 is then displayed in detail, for example, using the several views depicted and described herein.

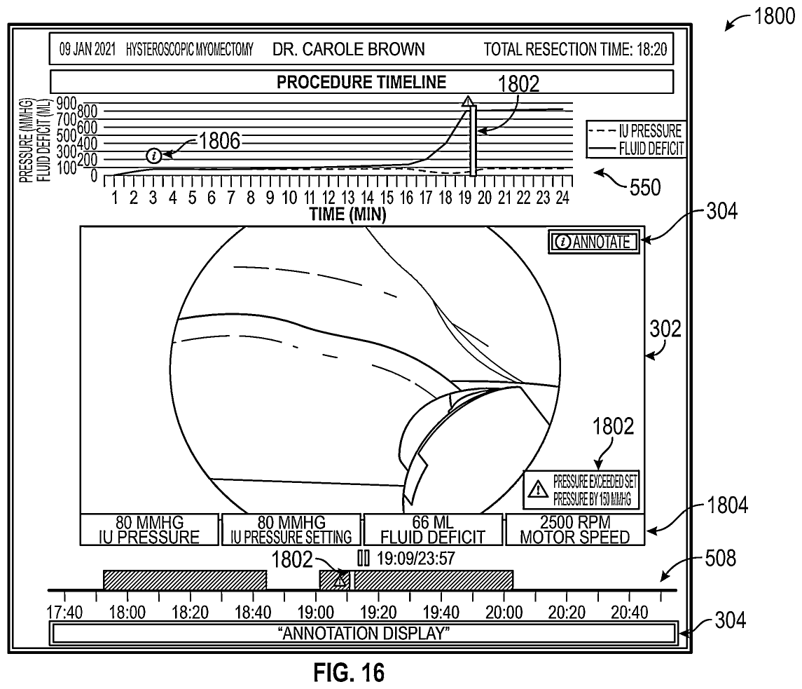

FIG. 16 depicts another view 1800 of the surgical data associated with the surgical procedure of the selected entry 1610. The view can include the video playback 302, playback timeline 508, and a procedure timeline 550. Here, the procedure timeline 550 represents values of one or more attributes as measured during the surgical procedure. For example, the attribute can include a measurement from the surgical instrument(s), for example, IU pressure. The procedure timeline 550 can further include a detected attribute, for example, fluid deficit, during the surgical procedure. It is understood that other attributes, e.g., motor speed, can be alternatively, or additionally, depicted on the procedure timeline 550. In some aspects, the present value of the one or more attributes are also displayed via a user interface element 1804.

The two or more values that are depicted on the procedure timeline 550 can be related to each other, for example, to calculate or determine a quality metric of the surgical procedure, or an event associated with the surgical procedure. For example, the IU pressure and the fluid deficit can be used to determine whether a pressure setting was exceeded. Alternatively, or in addition, a condition can be determined based on a single attribute that is depicted.

When a specific condition with any one or a combination of the depicted attributes is identified, a visual representation 1802 is depicted in both the procedure timeline 550 and the playback timeline 508. In some aspects, the video playback 302 is augmented to depict the visual representation 1802 indicative of the detected condition. The user can select the representation 1802 and in response, initiate playback of the video 302 to the timepoint where the condition occurs during the surgical procedure.

Further, the user can add annotations to the surgical procedure data while reviewing the surgical data via the view 1800. The annotations can be added using the annotations element 304. In response to an annotation being added, a visual representation 1806 is added to the procedure timeline, which when interacted with can display the annotation added. The visual representation 1806 can be added at a timepoint on the procedure timeline 554 indicative of the time in the surgical procedure for which the observation of the annotation was made.

The reports/views/annotations and other information described herein is added to an electronic medical record (EMR) in one or more cases. In some aspects, the information about specific surgical procedures can be stored in the patient record associated with the patient that was operated upon during the surgical procedure. Alternatively, or in addition, the information is stored in a separate database for later retrieval. The retrieval can be associated with the patient's unique identification, such as EMR-identification, social security number, or any other unique identifier. The stored data can be used to generate patient-specific reports.

The technical solutions described herein facilitate improvement in the performance of a surgical action, such as sealing by identifying to the actors, cases where seal dimensionality reduction could have been performed in the past. Technical solutions herein can also identify to an actor, such as a first surgeon, all instances of a surgical action (e.g., sealing) performed s/he performed in a surgical procedure and a comparison of the number of the same surgical actions performed by other surgeons. The first surgeon can interactively see the surgical actions being performed by himself/herself, and the other surgeons and determine improvements. For example, the first surgeon can observe ranges of electrical variable for various procedures and uses of the surgical instruments by other surgeons, and emulate such protocols.

Additionally, the technical solutions herein provide a convenient and practical application to track the training of one or more actors who are training to perform one or more surgical procedures.

In addition, the technical solutions described herein can facilitate the service provider (e.g., manufacturer of the CAS system, surgical instruments, etc.) to determine the typical range of electrical variables used across various surgical actions, phases, surgical procedures, etc. and calibrate the CAS systems, surgical instruments, etc. accordingly.

The technical solutions described herein can further facilitate comparing hospital quality care, surgeons, etc.

The examples described herein can be performed using a computer such as a server computer, a desktop computer, a tablet computer, etc. In one or more examples the technical solutions herein can be implemented using cloud computing technology.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system comprising:
a memory device; and
one or more processors coupled with the memory device, the one or more processors configured to:
determine, autonomously, one or more phases in a surgical procedure based on a video stream of the surgical procedure;
identify one or more usages of a surgical instrument used during the surgical procedure; and
display a chart of the one or more usages, wherein the chart divides the one or more usages according to the one or more phases respectively, and a representation of each of the one or more usages indicates a duration of each usage.

2. The system of claim 1, wherein the video stream of the surgical procedure is analyzed by a first device to determine and output the one or more phases in the surgical procedure and wherein the one or more usages of the surgical instrument are identified by a second device based on electrical energy applied to the surgical instrument.

3. The system of claim 1 or claim 2, wherein the one or more usages are identified based on an amount of electrical energy provided to the surgical instrument.

4. The system of any preceding claim, wherein the video stream of the surgical procedure captured by an endoscopic camera from inside a body of a subject of the surgical procedure.

5. The system of any preceding claim, wherein a visual attribute of the representation of each of the one or more usages is based on a type of the one or more usages.

6. The system of any preceding claim, wherein a type of the one or more usages is selected from a group consisting of energy activation, reloading, firing, incision, clamping, dividing, and stapling.

7. The system of any preceding claim, wherein the chart is user-interactive, and wherein an interaction with a first representation corresponding to a first usage displays a video segment of the surgical procedure comprising the first usage being performed.

8. The system of any preceding claim, wherein the one or more processors are further configured to playback the video stream of the surgical procedure, and wherein a user-interface element displays a timeline depicting one or more timepoints in the video stream at which the one or more usages are performed.

9. The system of claim 8, wherein the one or more timepoints are rendered based on a type of the one or more usages respectively.

10. The system of claim 8, wherein audio data corresponding to the one or more usages is generated artificially during the playback of the video stream.

11. The system of any preceding claim, wherein the one or more processors are further configured to:
display a list of the one or more phases in the surgical procedure, wherein an entry corresponding to a first phase from the one or more phases includes a user-interface element comprising a timeline depicting the one or more usages performed for the first phase.

12. The system of any preceding claim, wherein the representation of each of the one or more usages indicates a user that performed the usage.

13. The system of any preceding claim, wherein the representation depicts a comparison of usages performed by a first user and a second user.

14. The system of any preceding claim, wherein the representation of each of the one or more usages indicates an anatomical attribute of the subject of the surgical procedure, the anatomical attribute comprising a body mas index, a tissue thickness, and a gender.

15. A method comprising:
determining, autonomously, one or more phases in a surgical procedure based on a video stream of the surgical procedure;
identifying one or more usages of a surgical instrument used during the surgical procedure based on energy supplied to the surgical instrument; and
displaying a chart of the one or more usages and a user-interaction with a representation of each of the one or more usages causes a corresponding portion of the video stream to be played back.

16. The method of claim 15, wherein the chart groups the one or more usages according to the one or more phases respectively.

17. A computer program product comprising a memory device with computer-readable instructions stored thereon, wherein executing the computer-readable instructions by one or more processing units causes the one or more processing units to perform a method comprising:
determining, autonomously, a stapling being performed in a surgical procedure based on a video stream of the surgical procedure;
identifying one or more usages of a surgical stapler used during the surgical procedure based on energy supplied to the surgical stapler; and
displaying a chart of the one or more usages of the surgical stapler, wherein a user-interaction with a representation of each of the one or more usages causes a corresponding portion of the video stream with the use of the surgical stapler to be played back.

18. The computer program product of claim 17, wherein the chart displays an amount of energy used during each of the one or more usages of the surgical stapler.
